(19) United States
(12) Patent Application Publication
(10) Pub. No.: US 2012/0123288 A1
(43) Pub. Date: May 17, 2012

(54) METHOD AND APPARATUS OF DETERMINING EXHALED NITRIC OXIDE

(75) Inventors: Hans Willem Van Kesteren, Eindhoven (NL); Teunis Johannes Vink, Eindhoven (NL); Nicolaas Petrus Willard, Eindhoven (NL)

(73) Assignee: KONINKLIJKE PHILIPS ELECTRONICS N.V., EINDHOVEN (NL)

(21) Appl. No.: 13/383,591

(22) PCT Filed: Jul. 23, 2010
(86) PCT No.: PCT/IB2010/053361

§ 371 Date: Jan. 12, 2012

(30) Foreign Application Priority Data
Jul. 30, 2009 (EP) 09166814.5

(51) Int. Cl. A61B 5/087; A61B 5/08

(52) U.S. Cl. 600/532

(57) ABSTRACT

A method and apparatus of determining the level of exhaled nitric oxide (NO) is disclosed. The method involves measuring the level of exhaled NO (34) and the corresponding exhalation flow rate in one or more exhalations (30, 32) of a tidal breathing manoeuvre performed by a subject. The data is used with a model describing the flow dependence of exhaled NO to derive a value for exhaled NO corresponding to a fixed flow rate, especially to an exhaled NO level corresponding to a fixed flow rate of 50 ml/s. During the manoeuvre a variation in flow restriction (31) may be applied so as to vary the overall flow rate of exhalation. The method offers a simple and quick way to determine exhaled NO levels with good accuracy and is suitable for use with children.

### FIELD OF THE INVENTION

This invention relates to methods and apparatus for determining nitric oxide production in the lungs based on a tidal breathing manoeuvre and, in particular, to a methods and apparatus that is relatively quick and simple to perform or use and is suitable for use with children.

### BACKGROUND OF THE INVENTION

It is known that the concentration of nitric oxide (NO) in exhaled air can be used as an indicator of various pathological conditions. For instance the concentration of exhaled NO is a non-invasive marker for airway inflammation. Inflammation of the airways is typically present in people with asthma and monitoring for high concentrations of exhaled NO can be used in a test which is useful in identifying asthma. Further a measurement of exhaled NO can be used to monitor the effectiveness of inhaled corticosteroids in anti-inflammatory asthma management.

The standardized method of measuring exhaled NO requires a single exhalation test at a fixed exhalation flow rate of 50 ml/s for at least 10 seconds (or 6 seconds in children), at an overpressure of at least 5 cm H₂O. Recommendations on a standardized method by the American Thoracic Society and European Respiratory Society are set out in the paper “ATS/ERS Recommendations for Standardized Procedures for the Online and Offline Measurement of Exhaled Lower Respiratory Nitric Oxide and Nasal Nitric Oxide, 2005” American Journal of Respiratory and Critical Care Medicine Vol. 171, pp 912-930 2005.

Exhalation at a constant flow rate remains difficult or impossible for some adults and for younger children. As a consequence the U.S. FDA states that measurement of exhaled NO needs guidance by trained healthcare professionals and cannot be used with infants or by children under the age of 7. (FDA 510(k) summary NIOX MINO, Aerocrine AB).

Alternative breathing manoeuvres have been proposed, like tidal breathing, breath hold and multiple fixed flow exhalations. Patent application US2007/0282214 describes a method involving a series of single breath exhalations, wherein each exhalation is maintained at a constant flow rate but different flow rates are used for different exhalations. This method therefore requires the subject to maintain a series of different constant flow rates with a consequent increase in the complexity of performing the test. A further difficulty with these alternative procedures is that outcomes cannot readily be compared to the current standardized method.

### SUMMARY OF THE INVENTION

It is therefore an object of the invention to provide a method for measuring exhaled NO levels which mitigates at least some of the above mentioned disadvantages.

Thus according to the present invention there is provided a method of measuring exhaled nitric oxide comprising the steps of: taking a plurality of measurements of the level of nitric oxide in exhaled air and the corresponding exhalation flow rate obtained during a tidal breathing manoeuvre; applying said measurements to a model describing the flow dependence of exhaled nitric oxide; and using said model to derive a value of exhaled nitric oxide that corresponds to a fixed flow rate.

This method uses measurements obtained during a tidal breathing manoeuvre. Tidal breathing is a much more straightforward and natural breathing process and thus is much simpler for the subject to perform than tests using single breath exhalations at a fixed flow or tests requiring a breath hold for a certain time. Thus an exhaled NO measurement using a tidal breathing manoeuvre can be performed by the subjects themselves without guidance and probably in cooperative children from the age of three onwards. Tidal breathing generally involves a breathing frequency of 4-20 breaths per minute for adults and 20-40 breaths per minute for children and exhaled volumes of 300-1000 ml per breath for adults and 100-500 ml for children.

A tidal breathing measurement for exhaled NO has several significant differences to the standardized test. In the standardized exhaled NO measurement condition, i.e. a single breath manoeuvre at a flow rate of 50 ml/s, the exhaled NO is highly flow dependent. Consequently the flow rate in the standardized test needs to be controlled accurately, the guidelines require the flow rate to be controlled to within +/−10%. In the tidal breathing measurement no such specific control over flow rate is required and the test can therefore be easier to perform.

Exhalation flows during a tidal breathing manoeuvre are generally higher, typically ranging from 100 to 1000 ml/s, assuming breathing rates of 4-20 and 20-40 breaths per minute (for adults and children, respectively). The NO concentrations are lower at these higher flow rates which requires a higher sensitivity of the exhaled NO monitoring system. Furthermore, the device has to operate at a sufficiently high time resolution in order to capture the exhaled NO breathing profile during exhalation.

The method of this aspect of the invention uses measurements obtained during tidal breathing and applies the measurements to an appropriate model that describes NO production in the lungs. Each of the measurements consists of a measurement of the level of detected NO in the exhaled air, i.e. the concentration or amount of NO detected and the flow rate of the exhalation at or about the point that the NO measurement was taken. These measurements are used in a model describing the flow dependence of the exhaled nitric oxide to derive a value of exhaled NO corresponding to a fixed flow rate. In other words the tidal breathing measurements are translated into an exhaled NO level that corresponds to the level expected at the fixed flow rate.

Conveniently the value of exhaled NO derived corresponds to a fixed flow rate of or around 50 ml/s, for example a flow rate in the range of 45-55 ml/s. As explained previously a fixed flow rate of 50 ml/s is the current recommended and widely accepted standard by the American Thoracic Society (ATS) and European Respiratory Society (ERS). Therefore, an exhaled NO level corresponding to a fixed flow rate of 50 ml/s is currently used as a standard in assessing airway inflammation in asthma. The method of this aspect of the invention therefore provides a value for exhaled NO which is directly comparable to measurements obtained using the standardized procedure recommended by the ATS and ERS. However the value is obtained using measurements which are acquired using a tidal breathing manoeuvre.

Various different models of NO production and diffusion in the lungs are known and can be used as the model describing flow dependence of exhaled NO in the method of this aspect of the present invention. Key elements in these models are: i) a description of the geometry of the lung system, often in simplified compartment form, ii) NO generation, and iii) NO diffusion. For instance a two-compartment model of NO exchange dynamics represents the lungs by a rigid airway compartment and a flexible alveolar compartment—see for example Tsoukias et al. “A two-compartment model of pulmonary nitric oxide exchange dynamics” J. Appl. Physiol, Vol. 85, pp 653-999, 1998. A three compartment model is described in “Characterizing airway and alveolar nitric oxide exchange during tidal breathing using a three-compartment model”, P. Condorelli et al., J. Appl. Physiol. Vol 96, pp 1832-1842, 2004. Another model is a trumpet model with axial diffusion which takes account of the trumpet shape of the airways and assumes that axial diffusion is the dominating NO diffusion mechanism—see US2007/0282214 or P. Condorelli et al., J. Appl. Physiol. Vol 102,pp 417-425, 2007 for example. The trumpet model with axial diffusion can provide a good description of the flow dependent NO production in the lungs but is mathematically more complex than the two and three compartment models and requires use of approximate analytical solutions or numerical solutions. All the aforementioned models form approximations to a more general model describing the generation and transport of various gasses in the airway system by a partial differential convective diffusion equation where the 3-dimensional asymmetric airway structure is mapped into the flow through an axially symmetric tube with a varying diameter. The method may be applied using any appropriate model although currently a model including axial diffusion is preferred.

The models describing the flow dependence of exhaled NO generally are based on various flow independent parameters. The two compartment model for example has three flow independent parameters, the steady state alveolar concentration, the airway wall diffusing capacity and the airway wall concentration (alternatively, instead of the airway wall concentration parameter, the maximum airway wall flux of NO can also be used). The steady state alveolar concentration and airway wall concentration will vary with the severity of any airway inflammation whereas the airway wall diffusing capacity is a gas diffusion parameter related to the transfer of NO between airway wall and gas stream and differs only slightly for healthy and asthmatic people. The Condorelli approximation to the trumpet model with axial diffusion has three flow independent parameters, two of which vary with the severity of inflammation of the lungs, i.e. the steady state alveolar concentration and maximum airway wall flux of NO and one describing the axial gas diffusion. Although the flow-independent parameters linked to the severity of inflammation from the two-compartment model and axial diffusion dominated trumpet model have similar names their actual values are model dependent and values can only be converted in a simple way within a certain flow range.

The method involves using the measurements (i.e. the measurement of level of nitric oxide in exhaled air and the corresponding exhalation flow rate) to determine at least one flow independent parameter of the model which varies with inflammation severity. With an appropriate choice of model and related flow-independent parameters, just one such parameter enables a reasonable translation of the tidal breathing measurements to the exhaled NO level corresponding to a fixed flow and the method may therefore comprise determining only one flow independent parameter from said measurements. As described in more detail later use of a model where only one flow independent parameter needs to be determined means that measurements obtained in a tidal breathing manoeuvre with no imposed flow restriction, or a small constant flow restriction, can be used to determine the one parameter.

The model may conveniently be a model incorporating axial diffusion because inflammation severity is mainly linked to the maximum airway wall flux parameter and the steady state alveolar concentration is small in contrast to models neglecting axial diffusion. Thus in a model which incorporates axial diffusion the steady state alveolar concentration parameter may either be neglected or set at some constant value. Thus the method may comprising using a model with a constant or no contribution for a steady state alveolar NO concentration.

The method may involve setting at least one of the parameters of the model related to gas diffusion and/or at least one other parameter related to inflammation to a constant value. At least one of the parameters may be set to population average values, i.e. an average value that has previously been determined for the population. For some parameters different population average values may exist based on gender, age, etc. and the appropriate value for the subject can be chosen. Setting these parameters to population averages obviously results in some inaccuracy but the inventors have found that sufficiently accurate values of exhaled NO level at the fixed flow rate may still be obtained. Additionally or alternatively at least one of the flow-independent parameters may be set to a personal value previously obtained or estimated for the particular test subject. The method may be used to monitor the daily or longer term variation in fixed-flow NO value for a particular test subject. A value for one or more flow independent parameters could be determined for the subject and used in all successive measurements.

The model therefore incorporates one or more flow independent parameters which vary with inflammation that are determined from the measurements of the level of exhaled NO and the corresponding flow rate. The remaining parameters are either set as constants, as a population average relevant for the subject or a previously estimated or determined value for the subject is used. Thus once the relevant flow-independent parameters have been determined the model can be used to provide a value for exhaled NO which corresponds to a fixed flow rate, especially a fixed flow rate of 50 ml/s. Conveniently the model has an analytical solution.

In one embodiment the flow dependence of the exhaled nitric oxide C_{E} in the model is based on an analytical expression given by

$${C}_{E} = {C}_{alv} + \frac{{J}_{s}}{\dot{V}} {( 1 + \frac{{c}_{1} \cdot {D}_{aw}}{\dot{V}} )}^{- {c}_{2}} {( 1 + \frac{{c}_{3} \cdot {D}_{ax}}{\dot{V}} )}^{- c 4} \qquad Eqn . 1$$

where \dot{V} denotes the flow rate of exhaled air, D_{aw} the airway wall diffusion coefficient and Dₐₓ the axial diffusion constant for Nitric Oxide. Cₐₗᵥ is a flow independent contribution relating the steady state alveolar NO concentration. c₁, c₂, c₃ and c₄ are positive constants which are derived from fits to numerical solutions of the differential equation describing Nitric Oxide production, convection and diffusion in the airway tree. In one model c₁ may have a value of around 1 and c₂ may have a value of 0.4, c₃ may have a value around 2200 and c₄ may have a value of around 0.25.

J_{S} is a flow independent parameter which is particular to the subject and which is determined from the measurements of exhaled NO level and flow rate. The method therefore involves determining a value of J_{S} based on the measurements and then using this analytical expression to find a value for exhaled nitric oxide C_{E} at a particular flow rate \dot{V}, for example 50 ml/s.

Conveniently the tidal breathing manoeuvre is performed without any significant flow restriction during the test. The tidal breathing manoeuvre may for instance be performed with no flow restriction imposed, other than any flow restriction which is inherent in the measurement apparatus. Alternatively the method may comprise obtained measurements with a small constant flow restriction. However, in some embodiments the method may use a plurality of measurements obtained during a tidal breathing manoeuvre involving varying a flow restriction applied to exhalation such that at least one of said measurements is obtained under different flow restriction conditions to at least one other measurement.

The combination of reduced levels of NO and relatively short measurement time involved in measuring NO levels during a tidal breathing manoeuvre means that noise from the measurement system can become significant, even for the best currently available detectors. Taking measurements from a large number of exhalations can improve the accuracy but involves a measurement time which is significantly longer than the standardized test.

One embodiment of the method of the present invention uses data acquired during a tidal breathing manoeuvre during which a variation in flow restriction is applied to the exhalation phase such that the level of NO in exhaled air, i.e. the amount or concentration of NO detected, is measured for at least two different flow restriction conditions. This can improve the accuracy of the resulting fixed flow value derived from the model and aid in the determination of more than one flow independent parameters of the appropriate model.

It should be noted that the method of the present invention does not require or seek to achieve a constant flow rate exhalation and, as the measuring is done during a tidal breathing manoeuvre, the flow rate will likely vary throughout each exhalation. However varying a flow restriction will have a consequential effect on flow rates during an exhalation, i.e. if two tidal breathing exhalations from the same subject occur with different flow restrictions, say the first exhalation having a greater flow restriction applied than that applied for the second exhalation, both exhalations will have a variation in range of flow rates but the average flow rate during the first exhalation will be lower than that during the second exhalation.

Thus varying the flow restriction applied to exhalation in a tidal breathing manoeuvre will result in a general flow variation during exhalation. As mentioned above the detection of NO levels is dependent on flow rate and hence imposing a variation in the flow rates will result in a consequent variation in the measured exhaled NO levels.

This induced variation in flow rate and detected NO levels can aid in determining flow independent parameters of the model. Whilst normal tidal breathing does involve a variation in flow rates and hence detected NO levels the imposed flow restriction leads to a flow modulation which typically results in a greater range of flow rates and NO levels being measured. This greater range of detected NO levels can help improve the accuracy of the modelling and hence the derived value of the exhaled NO level corresponding to the fixed flow rate.

By applying the flow dependent model to a plurality of measurements acquired at different flow rates, an accuracy as good as that of the standardized test can be achieved, despite the much lower signal to noise ratio for each measurement due to the higher flow rates involved in tidal breathing.

It will be appreciated that, as mentioned above, during normal tidal breathing the flow rate will vary during exhalation. Thus several measurements taken during one or more tidal breathing exhalation without any variation in flow restriction would generally result in a range of NO values at different flow rates being obtained which can be sufficient to use in some models. However it has been found that, whilst the exhalation flow rate may vary relatively widely between individuals performing tidal breathing manoeuvres, an individual's flow range is relatively limited for most people. By applying a variable flow restriction the flow range for the individual is extended and additional data which can be used in the modelling is acquired.

The method may comprise the step of acquiring the data, i.e. getting the test subject to perform a tidal breathing manoeuvre and taking a plurality of measurements during the manoeuvre. During the tidal breathing manoeuvre a filter is conveniently used to remove NO from inhaled air as is standard in conventional measurements of exhaled NO.

Whether or not a variation in flow restriction is applied a relatively short tidal breathing manoeuvre may be performed. Conveniently the tidal breathing manoeuvre during which the NO levels are measured has a duration of a minute or less, i.e. the duration of the whole test is a minute or less. If a variation in flow restriction is imposed, it is imposed so that reasonable amounts of data can be acquired at each flow restriction condition.

As the method involves tidal breathing it is preferred that each measurement of NO level in exhaled air is acquired in a relatively short period so that it applies to a relatively constant exhalation flow part of the tidal breathing manoeuvre. Thus the method may involve using a NO detector with a time resolution which is sufficient to measure the NO patterns during tidal breathing, e.g. a sampling NO detector with a short sampling time. For instance the detector could be a chemiluminescent analyser such as is well known in the field of exhaled NO analysers. The method of this aspect of the present invention may therefore involve taking a plurality of measurements during each exhalation and may involve taking a plurality of measurements at each of the different flow restriction conditions.

Given no specified value of flow rate is required the method of the present invention can therefore be performed without any feedback regarding the flow rate being provided to the subject. The subject simply breathes as normally as possible through the measurement device. This makes the method of the present invention particularly suitable for use for children and means that the test can be performed without requiring specialist training of the person administering the test.

The tidal breathing manoeuvre is conveniently performed with a relatively low flow restriction applied to exhalation, i.e. no significant inhibition to exhalation. This again results in a natural breathing manoeuvre which is easily achievable by the majority of subjects including children. The tidal breathing manoeuvre may therefore include at least some measurements acquired during exhalation with an overpressure of 5 cm H₂O or less and conveniently an overpressure of 2 cm H₂O or less. An overpressure of 2 cm H₂O or less will be barely noticeable to most subjects and thus will not interfere with normal tidal breathing. Whilst the method does not require any particular flow restriction the use of apparatus to measure NO levels and flow rate, such as a mouthpiece with bacterial/viral filter or mask and blow tube for example, may inherently lead to some small flow restriction and hence some small overpressure.

When a variation in flow restriction is imposed at least one of the flow restriction conditions may involve a flow restriction which leads to an overpressure of 2 cm H₂O or less. Preferably each flow restriction condition gives rise to an overpressure of less than 5 cm H₂O or preferably 2 cm H₂O or less. Where a flow restriction is applied to the exhalation pathway this means that the maximum flow restriction must be sufficiently low so as to give rise to an overpressure of less than 5 cm H₂O or preferably 2 cm H₂O or less.

Having a relatively low overpressure for at least part of the tidal breathing manoeuvre means that for many subjects the velum may not be closed during exhalation. This leads to the possibility of contamination by air from the nasal cavity at the beginning of exhalation and the end of exhalation when the flow rate is low. Preferably therefore any measurements acquired during the start of an exhalation or the end of an exhalation are not used in the subsequent analysis. The method may involve only acquiring measurements of NO level during the middle of the exhalation phase but it may be simpler to acquire measurements throughout exhalation and subsequently disregard measurements acquired at the beginning and/or at the end of the exhalation. Measurements may also be disregarded when the breathing manoeuvre is interrupted for any reason such as a cough or choke action. Such interrupted exhalations could be noted by the subject or person administering the test. The remaining measurements can be seen as valid measurements and the period during which valid measurements are acquired is the total effective analysis time.

When a flow restriction is imposed during the tidal breathing manoeuvre, to ensure a flow modulation, the magnitude of the variation in the flow restriction may be arranged to give a significant variation in measured NO levels. By significant variation is meant a variation which is sufficiently greater than the NO detection error, taking the number of measurements or total effective analysis time into account, to give an accuracy which is at least comparable to that of the single breath, constant flow rate standardized test described above. As used herein the term total effective analysis time refers to the total time of each period during the tidal breathing manoeuvre during which measurements which can be used in a subsequent analysis are obtained. Thus the total effective analysis time is the sum total of each period during an exhalation where useable data is obtained. For an NO detector with a constant rate sampling time the total effective analysis time is effectively an indication of the number of measurements obtained.

In some embodiments the magnitude of the variation in flow restriction may be sufficient to cause a variation in measured NO levels which is significantly greater, say at least 25 times greater, than the NO detection error (1 σ) divided by the square root of the total effective analysis time.

The flow variation may be self imposed by the subject. As the method relates to tidal breathing and is not concerned about maintaining a flow rate at a constant, specified value, but rather achieving a relative change in flow rate, the subject could easily impose a change in the relative rate of exhalation. For instance the subject could breathe normally during one or more exhalations and also breathe with a self imposed reduction in flow rate during one or more other exhalations. In such an embodiment the method may comprise the step of measuring the flow rate and providing the subject with feedback regarding the current flow rate. An indication may provide the subject with guidance as to how to alter the flow rate, i.e. how much self-imposed flow variation to apply. The indication could comprise an indication to increase or decrease the flow rate or could indicate the current flow rate relative to an upper and/or lower threshold which is/are not to be crossed. The indication could indicate the actual or average flow rate or flow range of a previous exhalation with a view to the subject achieving a different flow rate or range in the current exhalation. The indication could be visible or audible or both.

Such indicators may be based on the indicators that are currently used in constant flow rate exhaled NO detectors. It will be appreciated however that the method of the present invention differs from the known methods in that the subject is not required to maintain a specific, constant flow rate.

Preferably however the step of varying a flow restriction comprises varying the flow restriction of the exhalation path of a measuring device.

It will be appreciated from the foregoing that imposing a variation in flow restriction does not necessarily require a flow restriction to be imposed in the exhalation path during each measurement. Thus the method may involve obtaining at least one measurement with no flow restriction applied, i.e. at least one measurement is obtained at a flow restriction condition where the condition is no applied restriction. Of course the use of an apparatus such as a mask/mouthpiece and blow tube to collect exhaled air for analysis may provide some minimal degree of flow restriction. However the degree of overpressure will be small and there will be no impediment to tidal breathing. Thus the step of varying the flow restriction in the exhalation path of a measuring device may comprise the step of introducing or removing a flow restriction into or from the exhalation path of a measurement device. This may be achieved by a variable flow restrictor being located in the exhalation pathway which can be varied so as to apply no flow restriction or could alternatively be achieved by switching a flow restriction element, which may for instance have a fixed flow restriction, into or out of the exhalation pathway.

The skilled person will be well aware of a variety of ways of varying the flow restriction of the exhalation pathway of a measuring device. For instance by opening and closing valves different exhalation pathways offering different levels of flow restriction could be selected. A variable flow restrictor, i.e. a device where the flow restriction can be varied could be used in the exhalation pathway. A constant flow restriction could be introduced into or removed from the exhalation pathway to provide the required variation.

Depending on the apparatus the flow restriction may be altered between discrete amounts of flow restriction, e.g. a valve is either open or closed, or may be variable in a continuous fashion.

It will be appreciated that varying a flow restriction in the exhalation pathway may be performed without providing any feedback regarding flow rate to the test subject.

A variation in flow restriction may be instigated manually or automatically. Where the flow restriction in the exhalation pathway is varied to provide the different flow restriction conditions, instigating the flow variation comprises applying the variation, either automatically by a controller or manually by the subject under test or by a person administering the test. In the embodiment where the flow restriction is self imposed instigating the variation comprises indicating to the subject that a flow rate change is required. This indication could be given, or altered, by a person administering the test or could be automatically provided.

The method may be arranged such that a variation in flow restriction is applied after a certain duration, for example the test could be started with one flow restriction condition and after a set time, say 20 s for example, the flow restriction varied to provide a second flow restriction condition. Conveniently however the variation in flow restriction is applied at the end of an exhalation or between exhalations. In other words one or more exhalations may be performed with a particular flow restriction and, at the end of an exhalation phase or during an inhalation phase, the flow restriction varied such that one or more subsequent exhalations occur with a different flow restriction. This could be achieved by the test subject, or person administering the test, applying a manual variation to the restriction of the exhalation pathway during an appropriate part of the tidal breathing manoeuvre. Similarly a person administering the test could alter the indication for a self imposed flow restriction at an appropriate point. Alternatively the flow restriction may be automatically applied between exhalations. The method may therefore involve detecting a period between exhalations, for instance by detecting the fast drop in flow rate at the end of an exhalation or a zero crossing indicating start of inhalation.

It will be appreciated from the foregoing that the preferred embodiment of the invention therefore involves measuring the NO levels of a plurality of exhalations of a tidal breathing manoeuvre.

The method may involve repeatedly varying the flow restriction between two or more flow restriction conditions, e.g. a first flow restriction condition may be imposed for one or more exhalations with a second flow restriction being imposed for one or more subsequent exhalations before returning to the first flow restriction condition. The method may also involve three or more different flow restriction conditions. The flow restriction may be varied between the respective flow restriction conditions in a predetermined pattern or sequence. For example the flow restriction could be varied between a first flow restriction condition and a second flow restriction condition between subsequent exhalations. If one flow restriction condition is a minimal flow restriction and the other is a higher flower restriction, this arrangement will result in exhalations with increased flow restriction being interspersed with exhalations with minimal restriction to maintain a natural breathing rhythm. In an alternative embodiment the flow restriction is varied between the different flow restriction conditions based on the detected pattern of breathing and thus is modified to match the current breathing. The flow restriction applied to an exhalation may be based, at least in part, on the magnitude of the average flow rate of one or more previous exhalations.

Additionally or alternatively at least one variation in flow restriction may be applied during a single exhalation so that two or more measurements are acquired under different flow restriction conditions during a single exhalation. Such a variation in flow restriction may involve one or more discrete changes in flow restriction or may involve a continuous variation in flow restriction over time.

In use, with different subjects, the same flow restriction could be applied to each subject. Thus a relatively simple apparatus could be used to apply the same variation in flow restriction for each subject or to indicate the amount of self-imposed flow restriction. However, it may be desirable to alter at least one of the flow restriction conditions and variation in flow restriction according to the average flow rate and or breathing pattern of the individual so as to ensure a suitable range for analysis.

The method of this aspect of the invention therefore provides a simple and easy method for determining exhaled NO which does not require the subject under test to perform any difficult or complicated breathing manoeuvres. The subject may simply breath normally in a tidal breathing manoeuvre. As mentioned this means that the invention may be used on a wide range of subjects, including children who may not be able to perform the standardized test. In some embodiments, imposing a flow modulation, through use of a flow restriction, can be used to increase the range of detected NO levels and flow rates which can be useful in determining the model parameters with greater accuracy.

It will be noted that the present invention relates to a method for determining the level of NO in exhaled air and thus relates to a technical method for measuring for a particular gaseous constituent. The method may be administered by a non-specialist non medically trained operator. The information regarding exhaled NO levels that is determined by the method may subsequently be used as a test or part of a test to identify a condition such as asthma and thus the method provides information that may be of assistance to a medically trained practitioner.

The method of taking the plurality of measurements and using an appropriate lung model to derive a value of exhaled NO corresponding to a fixed flow rate may be performed by a suitably programmed computer and, in another aspect of the present invention there is provided a computer program which, when run on a suitable computer or computer system and given the plurality of measurements as a data input, performs the method described above. The computer program may be stored on a computer readable storage medium.

The present invention also relates to a suitable apparatus and thus, in another aspect of the invention there is provided an apparatus for determining exhaled nitric oxide levels comprising: an exhalation pathway; a nitric oxide detector in fluid communication with the exhalation pathway and arranged to take a plurality of measurements of the level of nitric oxide in exhaled air; a flow rate detector in fluid communication with the exhalation pathway for taking a plurality of measurements of the exhalation flow rate; and a processor adapted to take the plurality of measurements of nitric oxide and exhalation flow rate obtained in a tidal breathing manoeuvre and derive a value of exhaled nitric oxide corresponding to a fixed flow rate.

The processor applies a model describing the flow dependence of the exhaled nitric oxide to the measurements obtained in a tidal breathing manoeuvre to derive the value of exhaled nitric oxide at a fixed flow rate. The processor may therefore be arranged to perform the method as described above with respect to the first aspect of the invention including any of the variants or embodiments of the method as described above. In particular the derived value of exhaled nitric oxide may correspond to a fixed flow rate of, or around, 50 ml/s so that the value is directly comparable to values obtained using the standardized single breath manoeuvre.

The processor may be arranged to determine the exhaled NO based on the analytical expression given as eqn. 1 above.

The apparatus may include a variable flow restrictor operable on the exhalation pathway. The variable flow restrictor can be used to apply a variation in flow restriction as described above. The variation may be manually imposed or may be automatically imposed. The apparatus may therefore comprise a flow restrictor controller, the controller being adapted to control the variable flow restrictor so to vary the flow restriction. The processor may be adapted to act as the flow restrictor controller. In one embodiment the controller is responsive to the flow rate detector so as to, in use, apply at least one variation in flow restriction at the end of an exhalation or between exhalations.

Additionally or alternatively the apparatus may include a feedback device, responsive to the flow detector, for providing feedback to the subject regarding the exhalation flow rate. The feedback device may comprise at least one visible display for displaying the current flow rate and/or at least one audio device for audibly indicating the current flow rate.

The apparatus may comprise a memory for maintaining personal data regarding one or more subjects. The personal data may comprise historical exhaled nitric oxide data, i.e. measurements recorded in one or more previous tests and/or nitric oxide values derived from such previous tests. The personal data may comprise one or more model parameters that have been derived or estimated for the particular test subject.

These and other aspects of the invention will be apparent from and further described with reference to the embodiments described hereinafter.

### BRIEF DESCRIPTION OF THE DRAWINGS

The present invention will now be described by way of example only with reference to the following drawings, of which:

FIGS. 1a and lb illustrate methods of determining exhaled NO according to embodiments of the method of the present invention;

FIGS. 2a and 2b illustrate further embodiments of methods of determining exhaled NO according to the present invention;

FIG. 3 shows measured flow rate, applied flow restriction and measured NO levels for two exhalations of a tidal breathing manoeuvre; and

FIG. 4 shows a plot of measured exhaled NO concentration against inverse flow rate.

FIG. 5 shows a comparison of 50 ml/s NO values derived from a tidal breathing manoeuvre with 50 ml/s values obtained in the standardized procedure.

### DETAILED DESCRIPTION OF EMBODIMENTS

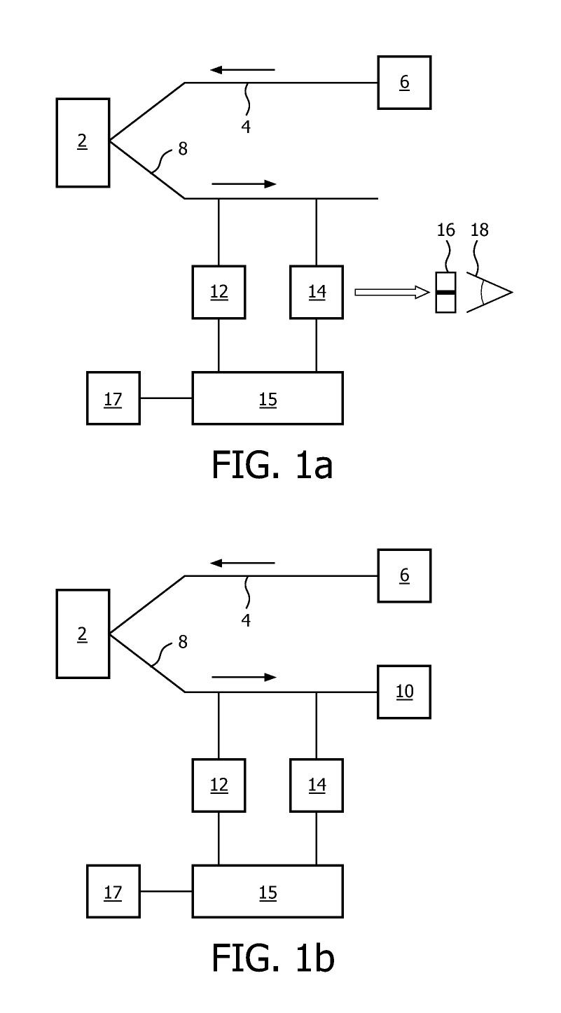

FIG. 1a illustrates a first embodiment of the present invention. A subject under test inhales and exhales via a mouthpiece 2. The mouthpiece may be a standard mouthpiece as used in current NO measuring equipment. During inhalation valves in the mouthpiece allows airflow along inhalation pathway 4 only and air is inhaled via an NO filter 6 to remove nitric oxide from the inhaled air. Subsequently the subject exhales, via exhalation pathway 8. It will be noted that exhalation pathway 8 has no flow restriction element associated with it and there is no impediment to exhalation other than the minimal restriction provided by the exhalation pathway.

An NO analyser 12, such as a currently available chemiluminescent NO analyser receives, at least some of the exhaled air and measures the NO concentration during this exhalation. Analyser 12 can also be based on a fast responding electrochemical NO sensor or a NO-to-NO₂ converter in combination with a photo-acoustic NO₂ sensor. In the latter case the filter unit 6 should be a NOₓ filter to remove both the NO and NO₂ from the inhaled air. Flow rate sensor 14, for instance a differential pressure sensor to measure a pressure drop over a minimal restriction in the exhalation path, also determines the flow rate during exhalation and records the flow rate for use in later analysis. Instead of measuring a pressure difference, flow rate sensor 14 can also be based on an ultrasonic sensor, in which case no restriction is required in the flow path. Flow rate sensor 14 may also monitor the flow rate of the inhalation pathway so as to improve analysis of the tidal breathing pattern. Note that depending on the different path lengths to the NO detector and the flow sensor, and any relative delay in operation of these detectors, there may be a need to apply a correction to the timings of the relative measurements to ensure that the measurements correspond to one another.

In use the subject breathes normally and NO levels and corresponding flow rates are recorded for one or more exhalations. Preferably measurements are acquired during a measurement period of about 30 seconds to one minute to enable enough NO and flow measurement data for the analysis.

In one embodiment of the present invention the subject simply breathes normally during the measurement period and the data on NO levels and flow rate is acquired and provided to a processor 15 for analysis. The processor may be arranged with memory 17 for storing data. If the subject under test has previously been tested the memory may store some personal information about that subject and may store various model parameters which are appropriate for that subject. The memory may be integrated with the processor or may be a removable memory apparatus.

In another embodiment however a general flow modulation is applied during the measurement period to increase the range of flow rate and NO levels detected. In the embodiment shown in FIG. 1a the flow modulation is self imposed by a subject.

A control display 16 indicates the flow rate to the subject 18 and also indicates how the flow rate should change during the tidal breathing manoeuvre. Conveniently the subject breathes normally for one or more exhalations and then self imposes a reduction in overall flow rate during one or more subsequent exhalations. The indicator could comprise a threshold not to be exceeded in the reduced flow rate condition or a target average value for example although the skilled person will appreciate that there are a number of ways a required variation in flow rate could be communicated to the subject.

In a simple embodiment only two flow rate conditions are required, e.g. normal flow rate and reduced flow rate, and all flow rates may include a substantial portion of the exhalation having a flow rate in excess of 100 ml/s. Several exhalations may be performed at both flow restriction conditions but the overall duration of the test may be less than a minute.

FIG. 1b illustrates an alternative arrangement where a flow restrictor is used to impose a slightly reduced exhalation flow rate or to provide a variation in flow rate. The apparatus is similar to that shown in FIG. 1a but in this case there is a flow restrictor 10 included in the exhalation pathway. A small flow restriction will result in a slight decrease in exhalation flow rates and hence a consequential increase in detected exhaled NO levels. Thus imposing a slight flow restriction may be used to increase the measurement accuracy or limit the time required for the tidal breathing manoeuvre. The restriction should be kept sufficiently small so as not to hinder the tidal breathing. Flow restrictor 10 may also be capable of varying the degree of flow restriction it imposes. It may be arranged in such a way that in one configuration it imposes no flow restriction and in another configuration it imposes a small flow restriction.

For example, a variable flow restrictor may be set to a first flow restriction, for instance no flow restriction, either manually by the subject or a person administering the test, or automatically by a controller (not shown in FIG. 1b). The subject then begins breathing normally and, after a certain period of time, the variable flow restrictor changes the applied flow restriction, for instance to increase the flow restriction. The length of time between the variation in flow restriction may be fixed or, where the restriction is changed manually, the subject or person administering the test may change the flow restriction after a certain number of exhalations.

Note that in this arrangement no feedback is provided to the subject regarding current flow rate as no such feedback is needed.

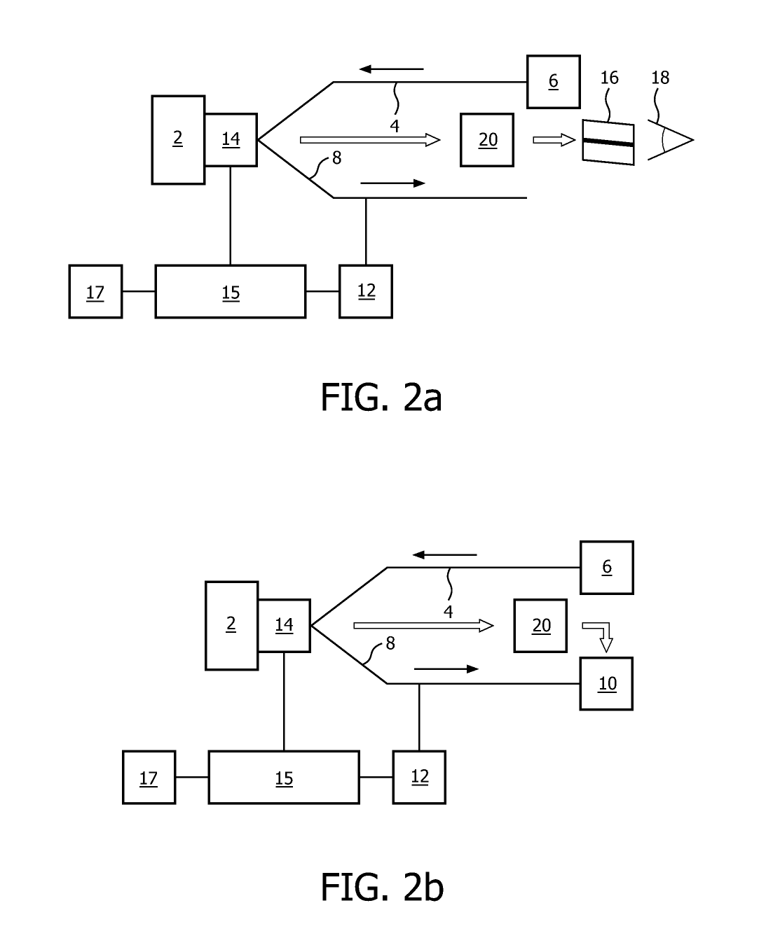

In other embodiments, as shown in FIGS. 2a and 2b, a variation in flow restriction may be adapted to the breathing pattern of the subject. FIG. 2a shows an arrangement similar to that shown in FIG. 1a but wherein the display 16 is controlled by an adaptive controller 20 which is responsive to flow sensor 14. Note in the embodiments shown in FIGS. 2a and 2b the flow rate sensor is illustrated as being integrated with the mouthpiece 2, which allows it to conveniently monitor flow rates of both the inhalation and exhalation pathways but other arrangements are possible.

The controller 20 determines, from the detected flow rates, the breathing patterns of the subject and determines an appropriate variation based thereon. This may involve determining the best time to indicate that the subject should impose a variation in flow rate and or determining how many exhalations should be performed at the reduced flow rate. Additionally or alternatively it may involve varying the indication of threshold flow rate or target average flow rate based on the measured flow rate.

FIG. 2b shows an arrangement similar to that shown in FIG. 1b but with a controller 20 controlling the variable flow restrictor 10 based on the measured flow rate. This adaptive flow controller enables adjustment of the timing of the flow restrictor changes to the breathing pattern and allows the optimization of the flow range to the subject's individual exhalation flow. The measured inhalation and exhalation flow is used to trigger a change in restriction setting. This can be done at the end of the exhalation when there is a fast flow reduction or at the zero crossing.

For all embodiments of the invention any flow restriction to exhalation can be relatively low. As mentioned above where the measurement period involves the subject breathing naturally for the whole period, i.e. without any variation in flow restriction, there could be no flow restriction in the exhalation pathway other than any minimal flow restriction imposed by use of the mouthpiece and a blow tube for example. For some embodiments an additional flow resistance may be present but preferably it imparts a low flow restriction so as to not significantly interfere with natural breathing. Even when there is a variation in flow restriction during the measurement period one flow restriction condition may comprise no flow restriction.

This means that the mouthpiece pressure experienced by the subject during exhalation may be less than 5 cm H₂O and more preferably less than 2 cm H₂O (98.067 Pa). This low level of pressure is preferred as it does not interfere with the normal tidal breathing manoeuvre. However, the relatively low pressure will mean that the subject's velum will typically stay open during the tidal breathing manoeuvre. Thus contamination by air from the nasal cavity, which usually has a high NO concentration, can occur around the transitions from inhalation to exhalation and exhalation to inhalation where the flow is low. During the middle part of the exhalation the flow is sufficiently high to prevent contamination.

The processor 15 therefore applies an analysis window to the measured NO profile to discard the possibly contaminated parts at the beginning and end phases of the exhalation. Furthermore, exhalations are excluded from the analysis that are disrupted, for instance by cough or choke actions. The total effective analysis time is the sum of the time spans for the analysis windows neglecting the disrupted exhalations.

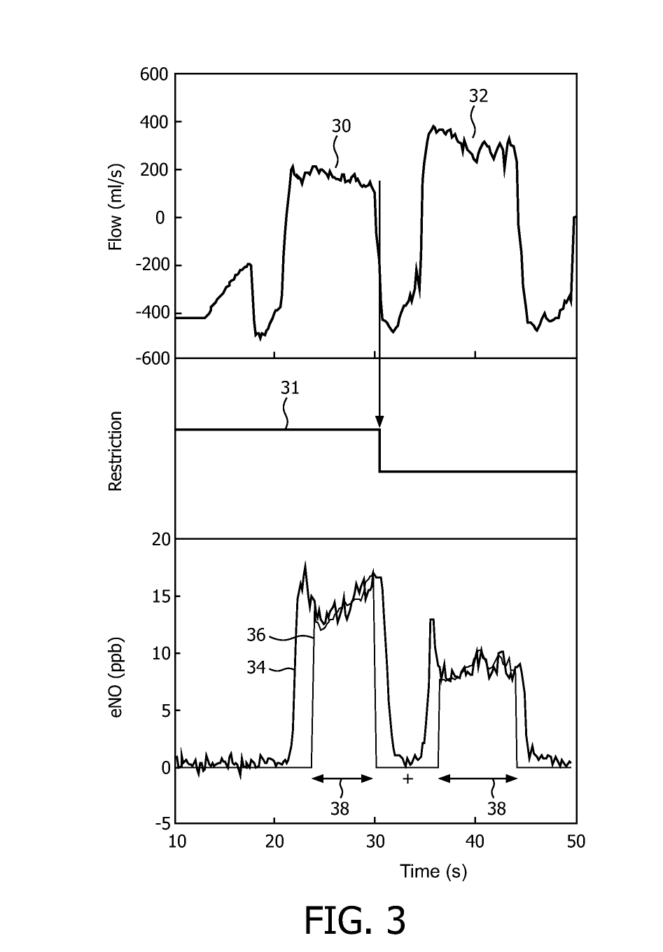

FIG. 3 illustrates the exhalation flow rate and the corresponding NO level for two exhalations where a flow restriction was imposed in the exhalation pathway. The figure shows the measured flow rate, the relative flow restriction applied and the measured concentrations of exhaled NO. FIG. 3 also illustrates the analysis windows applied to the two exhalations.

During a first exhalation 30a constant flow restriction 31 is applied which is reduced at the end of the exhalation. This reduction may be triggered by monitoring the breathing pattern and detecting a fast reduction in flow rate or zero crossing. The second exhalation 32, which occurs with a reduced flow restriction, can be seen to have a flow rate which has a greater average magnitude than the first exhalation. In both exhalations the flow rate varies throughout the exhalation. For the first exhalation 30, after a rapid increase at the start of exhalation, the flow rate varies from around 200 ml/s to 150 ml/s. For the second exhalation 32 the flow rate varies from nearly 400 ml/s to about 250 ml/s. Thus it will be clear that measurements at relatively high flow rates are obtained and there is a significant variation during the exhalation.

The effect on detected NO concentrations can be clearly seen. Line 34 indicates the data acquired by the detector and it can be seen that the measured concentration is significantly lower in the second exhalation than in the first exhalation. This is due to the higher flow rates of the second exhalations.

It can also be seen that the measured NO levels are generally higher at the start of an inhalation due to an increased residence time in the airways around the transition from inhalation to exhalation as well as nasal NO contamination in the low exhalation flow range. Nasal NO contamination can also occur at the end of the exhalation. Line 36 indicates the data after measurement windows have been applied to identify valid data and a fit has been applied. This identifies the exhalations, omits any data associated with the start or end of exhalation, and also omits any exhalations that have been interrupted by coughing or swallowing for example. The resulting windows 38 define the analysis time for each exhalation and the sum total of each analysis time gives the total effective analysis time, which is about 13 seconds in this example.

The two exhalations occur in a period of 30 seconds or so. Allowing a short time to achieve a normal tidal breathing rhythm the duration of the test can easily be of the order of 30 seconds to a minute or less.

Once the processor has identified the valid data the measurements are used to determine one or more flow independent parameters in a lung model.

Various lung models can be used. The two-compartment model of nitric oxide production in the lungs is well known, for instance as described by Tsoukias et al. in “A two compartment model of pulmonary nitric oxide exchange dynamics”. J. Appl Physiol 1998;85:653-666.

This two compartment model describes the lungs as a cylindrical rigid airway compartment with a volume of around 150 ml and a flexible alveolar compartment. The airway compartment is described by two parameters, the airway diffusing capacity and either the airway wall concentration of NO or maximum airway wall flux of NO. The alveolar compartment is described by a single parameter, the steady state alveolar concentration of NO. The exhaled NO concentration C_{E} as a function of the flow \dot{V} according to the two compartment model is given by:

C_{E}=C_{w}·(1−e^{-D}^{_{aw}}^{/\dot{V}})+Cₐₗᵥ·e^{-D}^{_{aw}}^{/\dot{V}} Eqn. 2

The exhaled NO concentration depends on the wall concentration C_{w} and alveolar concentration Cₐₗᵥ weighted by terms that depend on the airway diffusion coefficient D_{aw} and the flow. The analytical expression is valid for all relevant flows i.e. from above the tidal breathing regime to below 50 ml/s.

The limited flow range and relatively low signal to noise ratio of the detected NO levels at tidal breathing flow rates mean that a standard tidal breathing manoeuvre is not satisfactory for determining all three flow-independent parameters C_{w}, Cₐₗᵥ and D_{aw} for the two compartment model. In the determination of a 50 ml/s NO value from the tidal breathing data, C_{w} and Cₐₗᵥ form the most relevant parameters while an accurate value for D_{aw} is less important. In clinical studies it has been observed that the airway diffusing capacity is not directly related to inflammation severity. The airway diffusing capacity can be estimated based on population averages. In case the tidal breathing NO measurement device is used for repeated measurements of an already diagnosed asthmatic individual a population average value for asthmatic persons of D_{aw} can be used. Alternatively the airway diffusing capacity for a particular subject may have been derived previously, for instance from different measurements, and may be entered into the processor or obtained from memory 17.

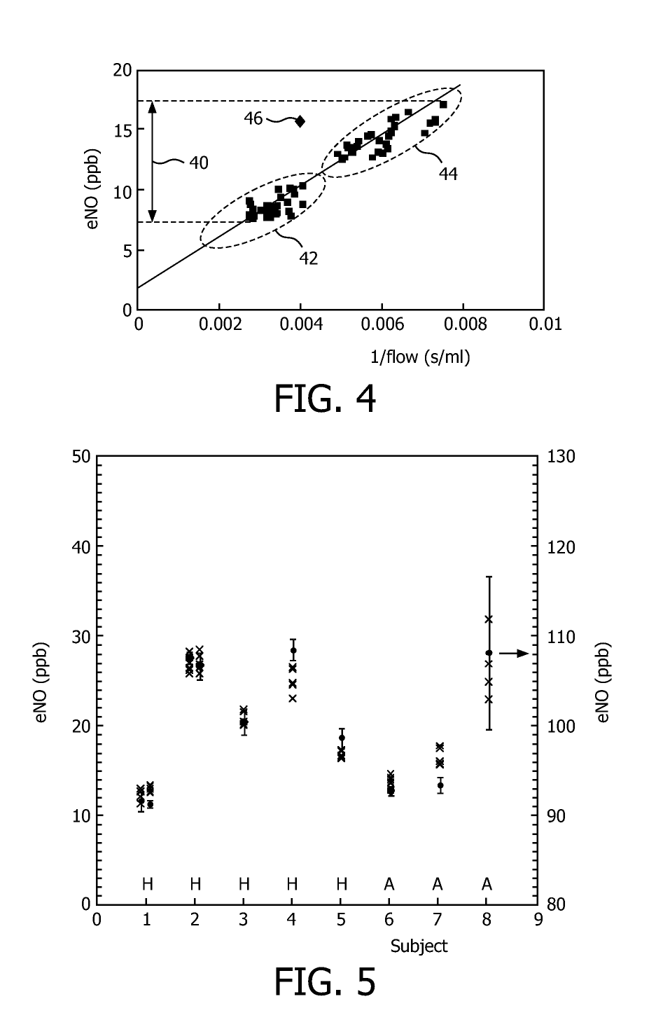

If a flow modulation is applied to a tidal breathing manoeuvre the variation in tidal breathing flow rates allows a reasonable estimate of the two inflammation related parameters C_{w} and Cₐₗᵥ to be made. FIG. 4 shows a scatter plot of the relevant NO and flow data points within the analysis window acquired during the exhalations shown in FIG. 3, as well as a two-parameter fit based on the expression given above and a fixed D_{aw} to derive C_{w} and Cₐₗᵥ. Data points corresponding to the first exhalation, where the higher flow restriction was imposed, are illustrated as group 44. Group 42 illustrates the data points for the second exhalation with the lower flow restrictions. The total range in detected NO concentrations is shown by arrow 40 and corresponds to a variation in NO concentration of nearly 10 ppb.

This figure indicates that the exhaled NO measurement range 40 is significantly larger than the 1 σ NO measurement error 46, so the procedure conforms to a proposed condition that the variation in measured NO levels is preferably at least 25 times greater than the NO detection error (1 σ) divided by the square root of the total effective analysis time. The flow variation from around 300 to 150 ml/s is well within the easy-to-perform tidal range.

With the two inflammation related parameters derived from the data and the airway diffusion parameter either estimated or obtained from another source, the analytic solution to the model can be applied to determine a value of exhaled NO that corresponds to a fixed flow rate of 50 ml/s or any other fixed flow rate. An exhaled NO level corresponding to a fixed flow rate of 50 ml/s is most useful since it corresponds with the current accepted ATS/ERS standard.

When the tidal breathing exhaled NO test is performed on a regular basis for a particular subject, the alveolar concentrations from a series of measurements can be stored in memory. These values can be taken into account in the two parameter fit for instance by applying a moving average to these earlier alveolar concentrations and the current one.

Whilst the two compartment model can give satisfactory results with data obtained with a sufficient variation in flow rate or a prior knowledge of the alveolar concentration, a model including axial diffusion is currently preferred as it can provide a sufficiently accurate description of the flow-dependent NO production in the flow range from tidal breathing down to 50 ml/s on the basis of one inflammation parameter only, which is the maximum airway wall flux of NO. Due to the close to zero value of the intrinsic steady state alveolar concentration in a model incorporating axial diffusion, a description with only the maximum airway wall NO flux provides a good basis for determining an NO value corresponding to a fixed flow rate, such as 50 ml/s, from a tidal breathing manoeuvre. The axial gas diffusion constant is to a large extent a general gas diffusion constant. Application of a model where the inflammation is described by one dominating parameter has the main advantage that a one-parameter fit to the tidal breathing data becomes possible and flow-modulation is not necessary. A small flow restriction or small flow-modulation during measurement might still be advantageous because it will result in sampling of slightly higher nitric oxide values (due to the slightly reduced flow rates) thereby increasing the accuracy.

The trumpet model as, for instance, described in US2007/0282214 is an example of a model including axial diffusion, maximum airway wall flux of NO and a steady state alveolar concentration. This model can be further extended to include the airway diffusing capacity and for instance a maximum airway wall flux of NO that depends on the axial position in the airway tree. It should be noted that the values of the maximum airway wall flux of NO, steady state alveolar NO concentration and NO diffusing capacity for a model including axial diffusion cannot be directly compared to their value in for instance a two-compartment model without axial diffusion.

Trumpet models including axial diffusion are defined by a differential equation, a source term describing the NO production, boundary conditions to the alveolar and mouth region and a description of the trumpet shape. A general solution is not known but a numerical solution can be obtained when all the parameter values are known. A determination of one or more parameter values from experimental data becomes only possible on basis of an approximate analytical solution or a time-consuming and complex iterative numerical procedure.

US2007/0282214 discloses a linear approximation of the flow-dependent NO production for a trumpet model including a maximum airway wall flux of NO, steady state alveolar value and axial diffusion. The linear approximation is valid in the flow-range of 100-250 ml/s. Most tidal breathing manoeuvres are within this flow range but for some subjects, tidal breathing involves higher flows. Also the approximation is not valid for a flow rate of 50 ml/s and so a determination of an NO value corresponding to a fixed flow rate of 50 ml/s using this approximation would have significant errors.

Comparing numerical solutions and analytical approximations for typical values for the trumpet model parameters, the present inventors have found that the following analytical expression describes the NO production C_{E} as a function of flow \dot{V} for flows of 25 ml/s and above quite accurately:

$${C}_{E} = {C}_{alv} + \frac{{J}_{aw}^{′}}{\dot{V}} {( 1 + \frac{{D}_{aw}}{\dot{V}} )}^{- 0.4} {( 1 + \frac{2200 \cdot {D}_{ax}}{\dot{V}} )}^{- 0.25} \qquad Eqn . 3$$

Here, Cₐₗᵥ denotes the steady state alveolar concentration, J′_{aw} the maximum airway wall NO flux, D_{aw} the airway wall diffusing capacity for NO and Dₐₓ the axial gas diffusion constant. For flows around 50 ml/s and above, and the time scale involved in tidal breathing, D_{aw} and the steady state alveolar value Cₐₗᵥ can be set to zero. As a typical value for Dₐₓ, 0.23 cm²/s can be taken (The Properties of Gases and Liquids, R C Reid et al., New York: McGraw-Hill, 1988). The approximated analytical solution given above is based on the finding that (a product) of diffusion terms of the form:

$${( 1 + \frac{{c}_{1} {D}_{i}}{\dot{V}} )}^{- {c}_{2}} \qquad Eqn . 4$$

with c₁ and c₂ constant, provide a good description of the flow dependent NO production in the trumpet shaped airway. An analytical approximation is very powerful in the analysis of the measurement data because it enables a simple and fast determination of one or more flow-independent parameter(s) from experimental data as well as a determination of the fixed flow NO value.

FIG. 5 shows results from 8 subjects including 5 healthy individuals (H) and 3 asthmatics (A). In the figure, 50 ml/s values derived from tidal breathing manoeuvres are compared with 50 ml/s values obtained according to the standard procedure. The circles and error bars denote the NO value and its standard deviation obtained from the standard fixed flow exhalation manoeuvre. The crosses denote results obtained from tidal breathing manoeuvres. Besides subject 8 for which the exhaled NO values are denoted on the right axis all other exhaled NO values or denoted on the left axis. The tidal breathing manoeuvre consisted of 5 breathing cycles. Air was inhaled through a NO removing filter and a restriction modulator was included in the exhalation flow path. The analysis is based on NO and flow samples collected during the last four exhalations in a window corresponding to an exhaled volume (time integrated flow) of 0.3 to 0.8 times the maximum volume reached during the exhalation. The first exhalation was discarded because it is often contaminated by earlier inhaled nasal NO or NO from environmental air which is not yet sufficiently removed in the alveolar part of the lungs. For the analysis a model was used which includes some aspects of the two-compartment model and some aspects of the trumpet model. The upper part of the airway is described by a rigid tube compartment characterized by a maximum-airway wall flux and airway diffusing capacity. The lower part of the airway and alveolar region are described by a second compartment characterized by a steady state alveolar value resulting from a maximum-airway wall flux and axial diffusion along the airway.

$${C}_{E} = \frac{{J}_{aw}^{′}}{{D}_{aw}} \cdot ( 1 - ( 1 - f ) {e}^{- {D}_{aw / \dot{V}}} ) \qquad Eqn . 5$$

Here, f denotes the ratio of the steady state alveolar concentration and maximum airway wall concentration. The value off can be derived from numerical solutions of the differential equation including axial diffusion. For the analysis a value of 0.01 for f and 10 ml/s for D_{aw} was used. The maximum airway wall flux J′_{aw} can easily be obtained from a one parameter fit of the NO versus flow or NO versus inverse flow data within the measurement windows.

It can be seen that use of the model produces values for exhaled NO at 50 ml/s which are very close to the actual values measured using the standardized fixed flow test for all subjects. This shows that an approach wherein data is acquired in a tidal breathing manoeuvre can be used to determine an accurate value for exhaled NO corresponding to a fixed flow rate of 50 mls/s. This allows the results of the test to be directly compared to results obtained using the standardized method and yet the data can be acquired in a natural breathing process suitable for the majority of adults and young children and which may be self-administered.

Although the approach based on a two-compartment model including effects of axial diffusion gives quite acceptable results, it should be clear that further refinements are possible. For instance, by including more realistic descriptions of the airway shape and NO source terms that have different magnitudes in different parts of the airway. Inclusion in the model of the time-response of the nitric oxide analyser will allow to broaden the measurement window and to obtain an accurate analysis within a reduced number of exhalations.

It should be noted that when a restriction modulation is applied it can take more complex forms than a simple two level variation. Instead of two different restriction levels, three or more levels can be used. It is also possible to vary the restriction in a continuous fashion during an exhalation.

Also the (average) magnitude of the exhalation flow in a previous exhalation can be taken into account in the setting of the restriction for a subsequent exhalation. This enables to take account of different exhalation flows for different subjects and guarantees an optimal flow range to be available in the analysis.

The examples and embodiments described above and as shown in the drawings are intended purely to illustrate the invention and the invention is not be construed as being limited to the arrangements describe above. Other variations to the disclosed embodiments can be effected by those skilled in the art in practising the claimed invention from a study of the drawings, the disclosure and the appended claims.

In the appended claims the words “comprising” and “comprise” does not exclude other elements or steps, and the indefinite article “a” or “an” does not exclude a plurality. Any reference signs in the description should not be construed as limiting their scope. A method claim reciting a series of steps in a certain order does not preclude those steps being performed in a different order unless expressly stated.

1. A method of measuring exhaled nitric oxide comprising the steps of:
taking a plurality of measurements of the level of nitric oxide in exhaled air and the corresponding exhalation flow rate obtained during a tidal breathing manoeuvre;
applying said measurements to a model describing the flow dependence of exhaled nitric oxide; and
using said model to derive a value of exhaled nitric oxide that corresponds to a fixed flow rate.

2. A method as claimed in claim 1 wherein the fixed flow rate corresponds to a flow rate of 50 ml/s.

3. A method as claimed in claim 1 wherein the step of applying said measurements to said model comprises using said measurements to determine at least one flow independent parameter of the said model.

4. A method as claimed in claim 3 wherein the method comprising determining only one flow independent parameter using said measurements.

5. A method as claimed in claim 1 wherein the model describing the flow dependence of the exhaled nitric oxide comprises at least one flow independent parameter related to gas diffusion which is preset at a constant value, a population average for the subject or previous personal value for the subject.

6. A method as claimed in claim 1 wherein the model describing the flow dependence of exhaled nitric oxide incorporates axial diffusion of nitric oxide and said model incorporates a constant or no contribution for a steady state alveolar NO concentration.

7. A method as claimed in claim 1 wherein the flow dependence of the exhaled nitric oxide C_{E} in the model is based on an analytical expression given by
$${C}_{E} = {C}_{alv} + \frac{{J}_{s}}{\dot{V}} {( 1 + \frac{{c}_{1} \cdot {D}_{aw}}{\dot{V}} )}^{- {c}_{2}} {( 1 + \frac{{c}_{3} \cdot {D}_{ax}}{\dot{V}} )}^{- c 4}$$
where \dot{V} denotes the flow rate, D_{aw} the airway wall diffusion coefficient and Dₐₓ the axial diffusion constant for Nitric Oxide, Cₐₗᵥ a flow independent contribution, and c₁,c₂, c₃ and c₄ positive constants and wherein J_{S} is a flow independent parameter which is determined from said measurements.

8. A method as claimed in claim 1 wherein said measurements are obtained during a tidal breathing manoeuvre that comprises varying a flow restriction applied to exhalation such that at least one of said measurements is obtained under different flow restriction conditions to at least one other measurement.

9. A method as claimed in claim 1 wherein said measurements are obtained during a tidal breathing manoeuvre with a self imposed flow variation such that at least one of said measurements is obtained under different flow conditions to at least one other measurement.

10. A method as claimed in claim 1 further comprising the step of disregarding measurements of nitric oxide which are acquired during at least one: the start of an exhalation; the end of an exhalation; the first exhalation of the tidal breathing manoeuvre; an interrupted exhalation; or where the flow rate drops below a predetermined threshold.

11. A computer program which, when run on a suitable computer or computer system, performs the method as claimed in claim 1.

12. An apparatus for determining exhaled nitric oxide levels comprising:
an exhalation pathway;
a nitric oxide detector in fluid communication with the exhalation pathway and arranged to take a plurality of measurements of the level of nitric oxide in exhaled air;
a flow rate detector in fluid communication with the exhalation pathway for taking a plurality of measurements of the exhalation flow rate; and
a processor adapted to take the plurality of measurements of nitric oxide and exhalation flow rate obtained in a tidal breathing manoeuvre and derive a value of exhaled nitric oxide corresponding to a fixed flow rate.

13. An apparatus as claimed in claim 12 wherein the derived value of exhaled nitric oxide corresponds to a fixed flow rate of 50 ml/s.

14. An apparatus as claimed in claim 12 wherein the apparatus comprises a memory for maintaining personal data regarding one or more subjects wherein the personal data comprises one or more model parameters that have been derived or estimated for the particular test subject.

15. An apparatus as claimed in claim 12 wherein the apparatus comprises a processor adapted to calculate the flow dependence of the exhaled nitric oxide C_{E} based on an analytical expression given by
$${C}_{E} = {C}_{alv} + \frac{{J}_{s}}{\dot{V}} {( 1 + \frac{{c}_{1} \cdot {D}_{aw}}{\dot{V}} )}^{- {c}_{2}} {( 1 + \frac{{c}_{3} \cdot {D}_{ax}}{\dot{V}} )}^{- c 4}$$
where \dot{V} denotes the flow rate, D_{aw} the airway wall diffusion coefficient and Dₐₓ the axial diffusion constant for Nitric Oxide, Cₐₗᵥ a flow independent contribution, and c₁,c₂, c₃ and c₄ positive constants and wherein J_{S} is a flow independent parameter which is determined from said measurements.
